(19) United States
(12) Patent Application Publication
(10) Pub. No.: US 2009/0318855 A1
(43) Pub. Date: Dec. 24, 2009

(54) DEVICES AND METHODS FOR IMPROVING INTRAVASCULAR UPTAKE OF AGENTS

(75) Inventors: Kevin J. Ehrenreich, San Francisco, CA (US); Kim-Lien Nguyen, San Francisco, CA (US); Randolf von Oepen, Los Altos, CA (US); Gregory W. Chan, San Francisco, CA (US); William Earl Webler, Jr., Escondido, CA (US); Travis R. Yribarren, Coarsegold, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: ABBOTT CARDIOVASCULAR SYSTEMS INC., Santa Clara, CA (US)

(21) Appl. No.: 12/145,212

(22) Filed: Jun. 24, 2008

(51) Int. Cl. A61M 1/00; A61M 25/10

(52) U.S. Cl. 604/28; 604/35

(57) ABSTRACT

This invention is generally related to therapeutic treatment of a coronary vessel wall, and more particularly, to systems and methods for improving uptake of therapeutic agents within the endothelium for enhanced delivery of therapeutic agents to a treatment site. A medical device is provided that is capable of reducing the pressure applied to a vessel wall and delivering a therapeutic agent into the vessel wall. Also, methods are described for using the device to facilitate delivery and uptake of a therapeutic agent into the vessel wall.

### BACKGROUND OF THE INVENTION

This invention is generally related to therapeutic treatment of a vessel wall, and more particularly, to systems and methods for improving uptake of therapeutic agents within a coronary vessel wall for enhanced delivery of therapeutic agents to a treatment site.

Percutaneous treatment of a coronary vessel wall at a treatment site, for regional therapy of vascular disease, includes delivery of a therapeutic agent into the coronary vessel wall. Delivery of therapeutic agents into the coronary vessel wall relies heavily on diffusion of the therapeutic agents through the endothelium into intercellular gaps. Delivery may occur, for example, by flushing the treatment area with a bolus of agent, or by bringing an agent-loaded surface into contact with the coronary vessel wall.

However, the effective migration of the therapeutic agent into the coronary vessel wall is limited by the anatomy of the channels within the vessel wall, and in particular by the size of those channels. Endothelial cell gaps and internal elastic lamina gaps are small relative to agent particles, and may prevent migration of these particles into the vessel wall. Therapeutic agents can be loaded into polymeric particles to provide a means for sustained drug delivery, but the endothelial gaps may also prevent delivery of these particles. Further, cellular gaps and agent migration may be affected by the pressure applied to the vessel wall by a medical device during agent delivery.

The effect of increasing transmural pressure may have an effect on vector delivery depth in the human vascular system. It is intuitive that an increase in transmural pressures may improve delivery into the vessel layers. However, there is scientific basis for an alternative hypothesis: that vector delivery can actually be improved by decreased transmural pressures.

This second hypothesis is based on a better understanding of the anatomy of the vascular wall. Specifically, the endothelium is comprised of a cellular matrix that on its surface includes cells connected but separated by endothelial cell gaps. Application of a radial load on the vascular wall results in tangential load conditions being applied to the vessel surface. As a result, the matrix may be distorted and the EC gaps may tend to close, thereby limiting the ability of the vessel to uptake particles that are above a certain size threshold.

In the context of these findings, existing technologies may not provide optimal solutions for delivering therapeutic agents into the vascular wall.

Devices for occluding a coronary vessel at two locations and delivering a therapeutic agent into the intermediate vascular space have been described, for example, in U.S. Pat. No. 7,060,051 to Palasis. In one aspect of the invention a multi-balloon catheter, such as a double-balloon catheter, with coating comprising a hydrogel polymer on the exterior surface of the occlusion balloons is provided. The balloons may be inflated inside the lumen to occlude a plaque-affected segment of the lumen thereby forming a lumen space between the balloons, wherein this plaque-affected lumen segment may be treated with a biologically active agent. Introduction of the biologically active agent may increase the vascular pressure above normal and may reduce the potential for uptake of the biologically active agents within the targeted vessel wall. The device design neither intends nor is capable of reducing the pressure applied to the vessel wall while delivering a therapeutic agent thereto, given that the catheter ports are not independent. In part, this is because the catheter design requires that fluid be moved through the ports simultaneously in the same direction.

Medical devices comprising catheters capable of establishing a flow of fluid between individual windows of a catheter body have also been described, for example, in U.S. Pat. No. 6,945,951 to Bonnette et al. This patent describes a cross stream thrombectomy catheter and system for fragmentation and removal of thrombus or other material from blood vessels or other body cavities. High velocity saline jets emitted from a toroidal loop jet emanator or other jet emanator in a catheter distal end entrain fluid through inflow orifices, and with flow resistances create a back-pressure which drives cross stream streams through outflow orifices in a radial direction and thence radially and circumferentially to apply normal and drag forces on thrombotic deposits or lesions in the blood vessel or other body cavity, thereby breaking apart and transporting thrombus particles to be entrained through the inflow orifices, whereupon the high velocity jets macerate the thrombus particles which then transit an exhaust lumen or recirculate again via the outflow orifices. The fluid flow generated by these types of devices is high enough to macerate thrombus and evacuate it from the patient anatomy. As a result, these types of device are neither intended nor are they capable of delivering biologically active agents into the coronary vessel wall.

Delivery of therapeutic agents into the coronary vessel wall may also be accomplished by utilizing drug-eluting stents and balloons, including the deployment of a medical device coated with a therapeutic agent at the treatment site. The therapeutic agent then migrates into the coronary vessel wall to provide the desired benefit. However, there concerns remain about the possibility of restenosis and the potential need for long to medium-term drug regiment to prevent complications when using these devices.

Therefore, a need exists to provide systems and methods for enhancing the delivery of therapeutic agents to a treatment site in a coronary vessel wall while minimizing or eliminating the occurrence of restenosis.

### SUMMARY OF THE INVENTION

The purpose and advantages of the present invention will be described and apparent from the description that follows, and through the practice of the invention.

To achieve these purposes and advantages, and in accordance with the present invention, a medical device is provided that in one aspect is capable of reducing the pressure applied to a vessel wall and in another aspect is capable of delivering a therapeutic agent into the vessel wall.

In an exemplary embodiment of the present invention, a medical device is provided comprising a catheter body with a first and second independent lumen. Further, a first and second sealing member are disposed on the catheter body such that the distal ends of the independent lumens are located between the sealing members. The independent lumens are capable of delivering a fluid such as a therapeutic agent or blood from a proximal end to a distal end, and vice versa.

In further accordance with the present invention, the first and second sealing members may comprise balloon components that are inflatable via inflation lumens.

In still further accordance with the present invention, the first and second sealing members may comprise expandable mechanical structures having a membrane covering capable of forming a fluid tight seal against a vessel wall.

In a further aspect of the present invention, a method is provided for delivering a therapeutic agent into a vessel wall. The method includes delivering the medical device into the target vessel section of a patient anatomy. The vessel section can be flushed and occluded by expanding the sealing members into contact with the vessel wall and delivering a flushing agent through the first independent lumen. A therapeutic agent can be delivered into the occluded vessel segment through the second independent lumen. The pressure acting on the vessel wall within the occluded vessel segment is reduced by removing fluid through the first independent lumen, causing a nontraumatic suction on the vessel wall. After reducing the pressure of the vessel segment, therapeutic agent can be introduced through the second independent lumen into the occluded vessel segment where it will be taken up by the vessel wall and administered into the layers of the vessel wall. The reduction of pressure applied to the vessel wall and administration of therapeutic agent may be repeated as necessary to achieve the desired distribution of therapeutic agent within the target vessel wall.

In an alternative embodiment of the present invention, a medical device is provided comprising a catheter body with a proximal and distal portion. An agent delivery member is disposed in the catheter body and is in fluid communication with an inflation lumen. An agent delivery lumen is disposed within the catheter body and is capable of delivering a therapeutic agent through the agent delivery member. A first and second sealing member are disposed distal and proximal to the agent delivery member such that expansion of the sealing members can occlude a vessel segment containing at least a portion of the agent delivery member.

In further accordance with the present invention, the first and second sealing members may comprise balloon components that are inflatable via inflation lumens.

In still further accordance with the present invention, the first and second sealing members may comprise expandable mechanical structures having a membrane covering capable of forming a fluid-tight seal with a vessel wall.

### BRIEF DESCRIPTION OF THE DRAWINGS

For a further appreciation of the above and other advantages, reference is made to the following detailed description and to the drawings, in which:

FIG. 1 is a plan view of an exemplary embodiment of a medical device for delivering a therapeutic agent into a vessel wall in accordance with the present invention;

FIG. 2 is a cross-sectional view taken about line A-A of FIG. 1 illustrating a catheter body construction in accordance with the present invention;

FIG. 3 is a partial plan view of a distal portion of an exemplary embodiment of a medical device disposed within a vessel segment in accordance with the present invention;

FIG. 4 is partial plan view of a distal portion of an exemplary embodiment of a medical device reducing the pressure applied to a vessel segment in accordance with the present invention;

FIG. 5 is a plan view of an alternative embodiment of a medical device for delivering a therapeutic agent into a vessel wall in accordance with the present invention;

FIG. 6 is a partial cross-sectional view taken about line B-B of FIG. 5 illustrating a distal portion of an alternative embodiment of a medical device for delivering a therapeutic agent into a vessel wall in accordance with the present invention;

FIG. 6A is a cross-sectional view taken about line C-C of FIG. 6 illustrating a catheter body construction in accordance with the present invention;

FIG. 7 is partial plan view of a distal portion of an exemplary embodiment of a medical device reducing the pressure applied to a vessel segment in accordance with the present invention;

FIG. 8 is partial plan view of a distal portion of an exemplary embodiment of a medical device delivering a therapeutic agent into a vessel segment in accordance with the present invention;

FIG. 9 is a plan view of an alternative embodiment of a medical device for delivering a therapeutic agent into a vessel wall in accordance with the present invention;

FIG. 10 is a plan view of an alternative embodiment of a medical device for delivering a therapeutic agent into a vessel wall in accordance with the present invention; and

FIG. 11 is a cross-sectional view taken about line D-D of FIG. 10 illustrating a catheter body construction in accordance with an alternative embodiment of the present invention.

### DETAILED DESCRIPTION OF THE PREFERRED EMBODIMENTS

In accordance with the present invention, a medical device is provided for delivering a therapeutic agent into a vessel wall. The medical device overcomes limitations of the vascular anatomy by reducing the pressure applied to the vessel wall before delivering a therapeutic agent into it. In this way, the cellular gaps that are the pathway for agent migration become more amenable to agent delivery and uptake of the therapeutic agent into the vessel wall is improved.

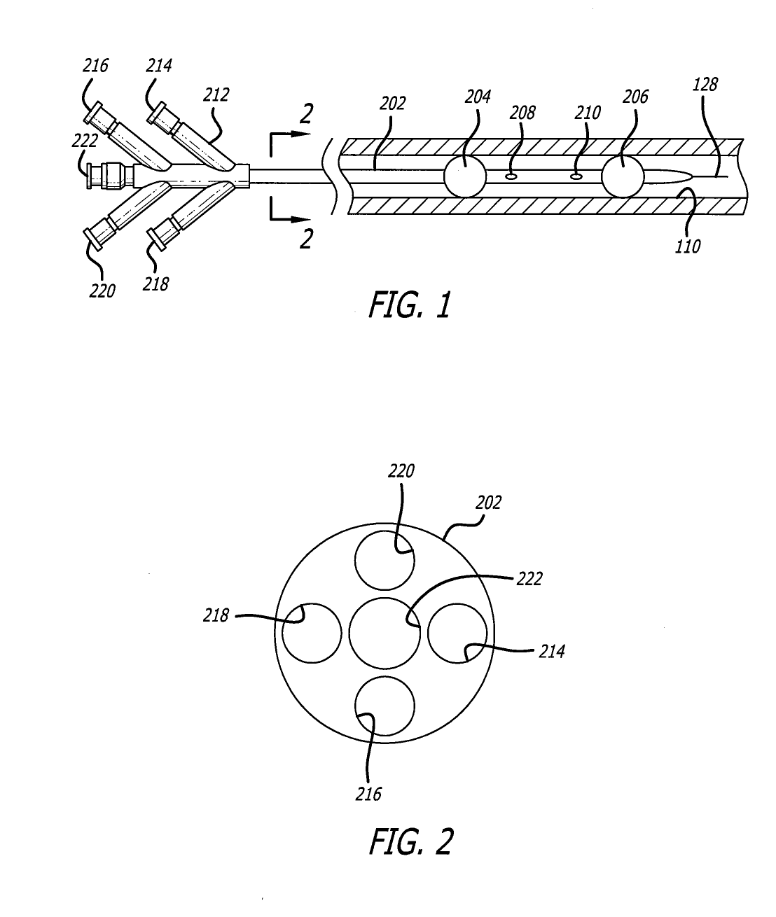

Referring now to FIG. 1, an exemplary embodiment of a therapeutic agent delivery device 200 is shown in accordance with the present invention. The device includes an elongated catheter body 202 with a proximal and distal end. A guidewire lumen 222 disposed within the catheter body 202 allows the device to be tracked over a guidewire 128 through a patient anatomy into a target vessel 110. The guidewire lumen 222 may have an over-the-wire design in which the proximal end of the guidewire lumen 222 is disposed within the lumen terminal 212, or it may have a rapid exchange design, in which the proximal end of the guidewire lumen 222 is disposed along the catheter body 202 distal to the lumen terminal 212. Both of these guidewire lumen designs are well known in the art of balloon catheters.

Near the distal end of the device is a distal portion and disposed on the distal portion is a proximal sealing member 204 and a distal sealing member 206. In this embodiment, the sealing members are inflatable balloon elements placed in fluid communication with independent first inflation lumen 214 and second inflation lumen 216. Each of these lumens have a proximal end disposed in the lumen terminal 212, which may be connected to a fluid source such as a syringe or inflation device to introduce or remove fluid from the sealing members.

The sealing members are placed proximal and distal to a proximal agent delivery port 208 and a distal agent delivery port 210 formed in the catheter body 202. These ports are located at the distal end of a first agent delivery lumen 218 and a second agent delivery lumen 220. These agent delivery lumens also have proximal ends disposed in the lumen terminal 212, which may be connected to a fluid source such as a syringe or inflation device to introduce or remove fluid from a vessel 110 in which the device is located.

Referring now to FIG. 2, a cross-sectional view is shown taken about line A-A of FIG. 1, illustrating an exemplary embodiment of a catheter body construction in accordance with this invention. The catheter body 202 comprises a first agent delivery lumen 218, a second agent delivery lumen 220, a first inflation lumen 214, a second inflation lumen 216, and a guidewire lumen 222. These lumens are sized and configured to be disposed within the catheter body 202 of the agent delivery device 200. It will be appreciated that this configuration may be modified in accordance with the present invention. For example, in the case of a rapid exchange guidewire lumen design, there may not be a guidewire lumen shown in this cross-section. Also, the lumen sizes may vary in accordance with this invention. Further, the lumen shapes may be configured differently. For example, one or more lumens may be have a non-circular cross-sectional appearance.

In a further aspect of the present invention, the agent delivery lumens may have different sizes in accordance with their function. In one embodiment the agent delivery lumen terminating in the proximal agent delivery port 208 can be larger than the other agent delivery lumen. This difference in size accommodates pressure losses over the length of the agent delivery lumens. Also, it complements the function of the proximal agent delivery port 208 in this embodiment, which receives fluid from the vessel 110 during the treatment procedure. Another consideration in determining the size of the agent delivery lumen is the viscosity of the fluid that is being delivered and retrieved from the vessel 110. As an example, the larger agent delivery lumen may have a diameter of between 600 and 700 micrometers and the smaller agent delivery lumen may have a diameter of between 400 and 600 micrometers.

The guidewire lumen 222 is sized and configured according to the size of the guidewire 128 that the agent delivery device 200 is to be used in combination with. For example, in the case of a typical coronary guidewire 128 with a diameter of 0.014-inches, the guidewire lumen 222 may have a diameter of approximately 0.016-inches.

In yet another aspect of the device, the inflation lumens can be sized to accommodate the viscosity of the inflation fluid and the pressure changes over the catheter length. In one example, the inflation lumens may have diameters of approximately 100 micrometers.

Although the size of the catheter body 202 can change depending upon the lumens that it accommodates, in a preferred embodiment the overall diameter of the catheter body 202 is approximately 1350 micrometers, making it well suited for intravascular procedures in accordance with the invention.

In further accordance with the present invention, a method of using the exemplary agent delivery device 200 to deliver a therapeutic agent and improve the uptake of the therapeutic agent within a vessel wall is provided. The agent delivery device 200 is tracked through a patient anatomy over a guidewire 128 to the target treatment site. Prior to deployment of the sealing members, all lumens of the device may be flushed with a fluid which does not interfere with the function of the therapeutic agent. The same or a different therapeutic agent, normal saline, heparin solution, or other fluids known or suitable may be used to flush the device lumens in accordance with this invention. The inflation lumens may be flushed with an inflation fluid such as saline, contrast fluid, or a solution thereof. The first agent delivery lumen 218 may be flushed with a flushing agent that does not interfere with the function of the therapeutic agent. The second agent delivery lumen 220 may be flushed with therapeutic agent to ease the timing of the procedure. This flushing procedure also aids in the prevention of introducing air into the vascular system, which can have detrimental effects.

The vessel 110 may be occluded and flushed. First an inflation fluid can be introduced through the first inflation lumen 214, thereby expanding the proximal sealing member 204 and bringing it into contact with the vessel 110. Subsequently a flushing agent can be introduced through the first agent delivery lumen 218 to flush the vessel distal to the proximal sealing member 204. An inflation fluid can be introduced through the second inflation lumen 216 to expand the distal sealing member 206, bringing the sealing member into contact with the vessel 110 and forming an occluded vessel segment 112.

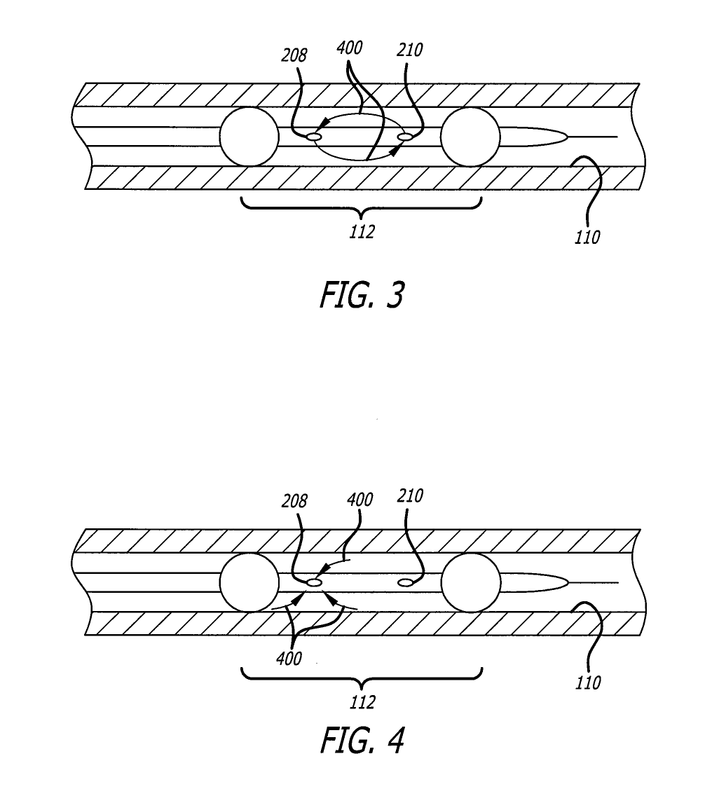

Referring to FIG. 3, after an occluded vessel segment 112 is established, an agent may be introduced into the occluded vessel segment 112 from the second agent delivery lumen 220 through the distal agent delivery port 210. In a preferred embodiment, the agent may have a viscosity lower than that of blood, making it easier to convey through the catheter lumens. For example, the agent may be saline, another non-therapeutic agent, or it may be a therapeutic agent. If the agent is a non-therapeutic agent, then it will be replaced with a therapeutic agent after the flushing procedure in order to facilitate the remainder of the procedure. The therapeutic agent can move passively or actively into the first agent delivery lumen 218 through the proximal agent delivery port 208. This fluid flow 400 may, for example, be facilitated by suction applied to the first agent delivery lumen 218.

In a preferred embodiment, the pressure within the occluded vessel segment 112 will not exceed about 200 mm Hg of pressure during the process of maintaining fluid flow 400. In a more preferred embodiment, fluid flow 400 will be introduced under a pressure of about 100 mm Hg. This should allow for flushing of the occluded vessel segment 112 of most of the remaining blood in a nontraumatic manner. By doing so, there is minimal risk of producing thrombus or injuring the vessel wall by over-pressurization.

Referring now to FIG. 4, the agent delivery device 200 is shown within the vessel 110 after having occluded and flushed the vessel 110 as described above. Fluid flow within the second agent delivery lumen 220 is stopped and fluid flow into the first agent delivery lumen 218 through the proximal agent delivery port 208 is maintained. This creates a reduction in fluid volume disposed within the occluded vessel segment 112 and results in a reduction in the pressure applied to the vessel wall. The removal of fluid through the first agent delivery lumen 218 should be continued until the intravascular pressure is reduced below the normal diastolic pressure of the patient. For example, it is contemplated that a reduction in intravascular pressure to approximately 10 mm Hg should be adequate. In a preferred embodiment, the removal of fluid through is facilitated by the reduction of pressure within the first agent delivery lumen 218 to below 0 mm Hg using a vacuum source, such as a syringe or vacuum pump.

As the intravascular pressure is reduced, the stress on the vessel wall will be reduced along with mechanical loads applied to the corresponding tissue. Intracellular gaps are likely to increase in size, especially in the endothelial cells and the internal elastic lamina leading into the media of the vessel wall.

When the pressure within the vessel wall reaches the intended lower pressure, therapeutic agent is introduced through the second agent delivery lumen 220. This increases the intravascular pressure above the lowest pressure and urge therapeutic agent into the vessel wall. In a preferred embodiment, the pressure is raised to an intermediate pressure between the lowest pressure reached within the vessel and the normal diastolic pressure of the patient. For example, the intravascular pressure may be raised to approximately 60 mm Hg. Since the pressure at which the therapeutic agent is delivered into the vessel wall is still below the normal diastolic pressure of the patient, the stress on the vessel wall will be comparatively reduced while the uptake of the therapeutic agent within the increased intracellular gaps will be improved.

Using the process described above, the intravascular pressure can be cycled between the lowest pressure and an intermediate pressure several times, allowing therapeutic agent to be taken up by the vessel wall in a pulsatile fashion. This will result in increased uptake of therapeutic agent as the intracellular gaps are opened and closed by the changes in intravascular pressure. Thus allowing the therapeutic agent to enter the vessel wall as the intracellular gaps widen and to be confined within the vessel wall as the intracellular gaps narrow. Each procedure may include from one to hundreds of cycles in accordance with this invention. In a preferred embodiment, there will be about five to fifteen cycles per procedure. In a more preferred embodiment, there will be about ten cycles per procedure.

Procedural length may vary based on the number of cycles and the duration of each cycle, in accordance with this invention. It is contemplated that the procedure would take place over a period of approximately 30 seconds to 1 minute, which is an understood and well-accepted period for occlusion-based therapies. In one embodiment, the procedure will take place over a period of about 30 seconds, wherein 10 cycles are performed as described above with each cycle lasting about 3 seconds; about half of the cycle being used to raise the vascular pressure and about half of the cycle being used to lower the vascular pressure. In an alternative embodiment, the procedure will take place over a period of about 60 seconds, wherein 10 cycles are performed as described above with each cycle lasting about 6 seconds; about half of the cycle being used to raise the vascular pressure and about half of the cycle being used to lower the vascular pressure. It will be appreciated that the procedural time, number of cycles, and duration of each cycle and cyclic stage can be varied in accordance with this invention. For example, each procedure may comprise hundreds of cycles that have an average duration of about a tenth of a second or less, or a single cycle with a duration of many seconds.

After a pre-determined number of cycles and agent delivery is complete, the proximal and distal sealing members 204, 206 can be deflated by removing inflation fluid through the inflation lumens. With the procedure being complete, the agent delivery device 200 and the guidewire 128 may then be removed from the patient anatomy.

Among the therapeutic agents that can be delivered into the vessel wall in accordance with this invention are anti-proliferative and cytostatic agents such as everolimus, ABT-578, tacrolimus and pimecrolimus, angiopeptin, calcium channel blockers such as nifedipine, amlodipine, cilnidipine, lercanidipine, benidipine, trifluperazine, diltiazem and verapamil, fibroblast growth factor antagonists, fish oil (omega 3-fatty acid), histamine antagonists, lovastatin, or various other agents known or suitable. In addition, topoisomerase inhibitors such as etoposide and topotecan, as well as antiestrogens such as tamoxifen can be used. Other therapeutic agents that are useful in accordance with this invention include anti-inflammatory, antineoplastic, antiplatelet, anti-coagulant, anti-fibrin, antithrombotic, antimitotic, antibiotic, antiallergic and antioxidant compounds. It will be appreciated that still other types of therapeutic agents may be delivered and taken up by a vessel wall by using the devices and methods described herein.

The therapeutic agent can also be dissolved within a solvent to further facilitate uptake within the vessel wall. For example, dissolving a drug within a substance such as DMSO, castor oil, or iodine contrast can increase the solubility of the solution, and therefore improve the ability of the drug to enter into the intracellular gaps.

The sealing members described above may be formed using balloon catheter constructions that are well known in the art. For example, they may be formed from compliant, semi-compliant, or non-compliant balloons. Preferably, the balloons will have a compliant construction, which limits the stress in the vascular wall and provides improved sealing characteristics for vessel occlusion. Typical balloon materials for forming these types of balloon members include silicone and thermoplastic elastomers (TPE), including vector, pellethan, synprene, and kraton. Alternative balloon member constructions such as semi-compliant or non-compliant balloons may be formed from polyvinyl chloride, polyethylene terephthalate, nylon, and Pebax, to name a few materials.

In an alternative embodiment, the occlusion balloons 604 and 606 may be replaced by sealing members comprising expandable members having a scaffold construction that are covered by a thin film of material to make the sealing members capable of forming a fluid tight seal against a vessel wall. It is contemplated that the scaffold construction may effectively be formed from materials that are well known in stent fabrication, such as shape memory alloys including nickel-titanium, copper-zinc-aluminum, and copper-aluminum-nickel, or deformable materials such as stainless steel, silver, platinum, tantalum, palladium, cobalt-chromium alloys such as L605, MP35N, or MP20N, niobium, iridium, any equivalents thereof, alloys thereof, and combinations thereof, or suitable plastics, or various other materials known or suitable. In a preferred embodiment, the film covering is elastomeric and could be formed from materials such as silicone and thermoplastic elastomers (TPE).

Construction of the catheter body and various lumens can be accomplished using catheter materials that are well known in the art. For example, the catheter components may be formed from materials such as nylon, urethane, polyurethane, polyvinylchloride, polyester, PEEK, PTFE, PVDF, Kyner, polyimide, polyethylene, or various other materials known or suitable. Coils and other supportive members such as stiffening elements or wires may be included within the catheter construction as needed to improve the performance and deliverability of the catheter within the scope of this invention.

An alternative embodiment of a medical device in accordance with the present invention may include only a single agent delivery port. In the exemplary device described above, multiple agent delivery ports were used to aid in the procedural timing and ease of the procedure, however, it is possible to use a single agent delivery lumen and port to flush the target region, remove fluid from the target region to reduce intravascular pressure therein, and introduce a therapeutic agent into the target region for delivery and uptake within the vessel wall.

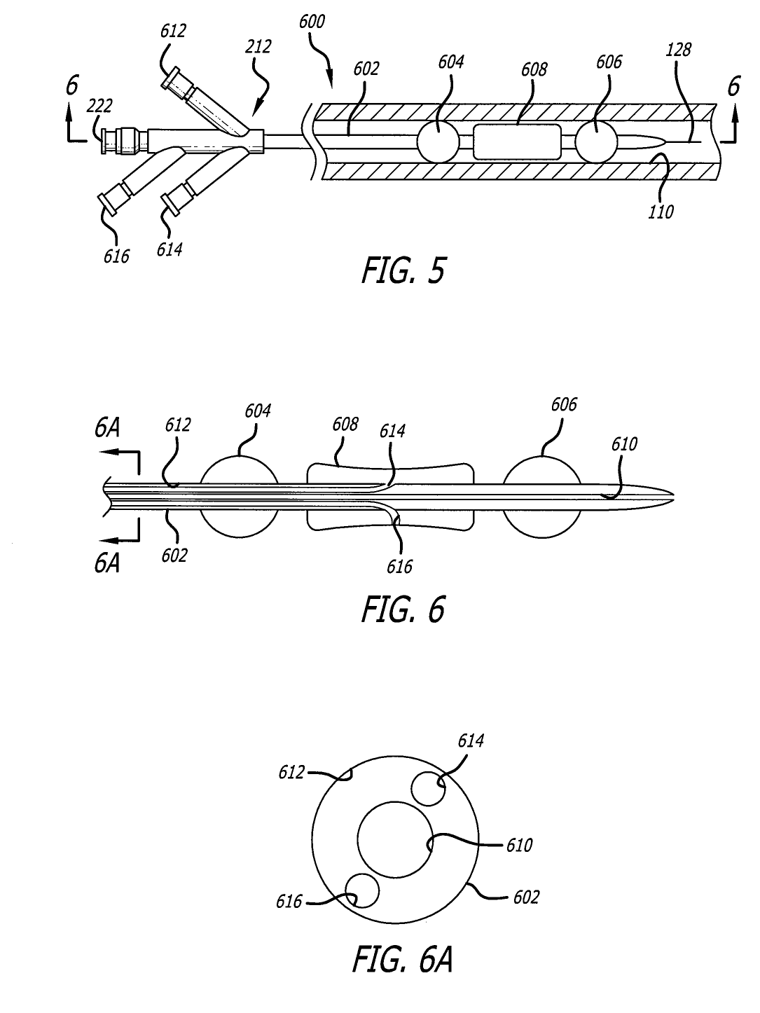

Referring to FIG. 5, an alternative embodiment of an agent delivery device 600 in accordance with the present invention is shown. The device includes an elongated catheter body 602 with a proximal and distal end. A guidewire lumen 610 (FIG. 6) may be disposed within the catheter body 602 to facilitate tracking the device over a guidewire 128 through a patient anatomy and into a target vessel 110. The guidewire lumen 610 may have an over-the-wire design in which the proximal end of the guidewire lumen 610 is disposed within the lumen terminal 212, or it may have a rapid exchange design, in which the proximal end of the guidewire lumen 610 is disposed along the catheter body 602 distal to the lumen terminal 212. Both of these guidewire lumen 610 designs are well known in the art of balloon catheters.

Near the distal end of the device is a distal portion and disposed on the distal portion is a proximal occlusion member and a distal occlusion member. In this embodiment, the occlusion members are inflatable balloon elements placed in fluid communication with occlusion balloon inflation lumen 612. This lumen has a proximal end disposed near the lumen terminal 212, which may be connected to a fluid source such as a syringe or inflation device to introduce or remove fluid from the occlusion members.

Referring now to FIG. 6, a cross-sectional view is shown taken about line B-B of FIG. 5, illustrating the distal portion of an alternative embodiment of an agent delivery device 600 in accordance with this embodiment of the invention. The occlusion members 604, 606 are placed proximal and distal to an agent delivery member 608 disposed on the catheter body 602. The agent delivery member 608 is in fluid communication with an agent delivery member inflation lumen 614 that terminates near a proximal end in the lumen terminal 212 (not shown).

An agent delivery lumen 616 is disposed within the catheter body 602 having a proximal end near the lumen terminal 212 (not shown) and a distal end disposed near the surface of the agent delivery member 608. The agent delivery lumen 616 passes through the agent delivery member 608 and may be formed separately or as part of the agent delivery member 608.

Figures depicts that the occlusion balloon inflation lumen 612 is placed in fluid communication with both the proximal occlusion balloon 604 and the distal occlusion balloon 606. This permits inflation and deflation of both occlusion members to be achieved simultaneously. It will be appreciated that it is also possible to include a second inflation lumen and to place each inflation lumen in fluid communication with a different occlusion member in accordance with this invention, thereby permitting individual inflation and deflation of the occlusion members if desired.

Referring now to FIG. 6A, a cross-sectional view is shown taken about section line C-C of FIG. 6, illustrating an alternative embodiment of a catheter body construction in accordance with this invention. The catheter body 602 comprises an agent delivery member inflation lumen 614, an agent delivery lumen 616, occlusion balloon inflation lumen 612, and a guidewire lumen 610. These lumens are sized and configured to be disposed within the catheter body 602 of the agent delivery device 600. It will be appreciated that this configuration may be modified in accordance with the present invention. For example, in the case of a rapid exchange guidewire lumen design, there may not be a guidewire lumen shown in this cross-section. Also, the lumen sizes may vary in accordance with this invention. Further, the lumen shapes may be configured differently. For example, one or more lumens may be have a non-circular cross-sectional appearance.

In further accordance with the present invention, a method of using an alternative agent delivery device 600 to deliver a therapeutic agent and improve the uptake of the therapeutic agent within a vessel wall is provided. The agent delivery device 600 is tracked through a patient's anatomy over a guidewire 128 to the target treatment site. Prior to deployment of the sealing members, all lumens of the device may be flushed with a fluid which does not interfere with the function of the therapeutic agent. The occlusion balloon inflation lumen 612 and the agent delivery member 608 inflation lumen may be flushed with an inflation fluid such as saline. The agent delivery lumen 616 may be flushed with a flushing agent that does not interfere with the function of the therapeutic agent, or it may be flushed with the therapeutic agent. This flushing procedure also prevents the introduction of air into the vascular system, which can have detrimental effects on the patient's health.

The agent delivery member 608 may be inflated by introducing an inflation fluid through the agent delivery member 608 inflation lumen. This will bring the agent delivery member 608 surface near the surface of the vessel 110. It will also serve the purpose of displacing most of the blood from the treatment site. A flushing fluid or a therapeutic agent may be delivered through the agent delivery lumen 616 while the agent delivery member 608 is inflated in order to further clear the target region of blood, if desired.

The proximal occlusion balloon 604 and the distal occlusion balloon 606 may be inflated by introducing an inflation fluid through the occlusion balloon inflation lumen 612. This will increase the profile of the occlusion balloons and bring them into contact with the vessel 110, thus establishing an occluded vessel segment 112. Alternatively, the inflation of the occlusion balloons may be completed prior to inflation of the agent delivery member 608.

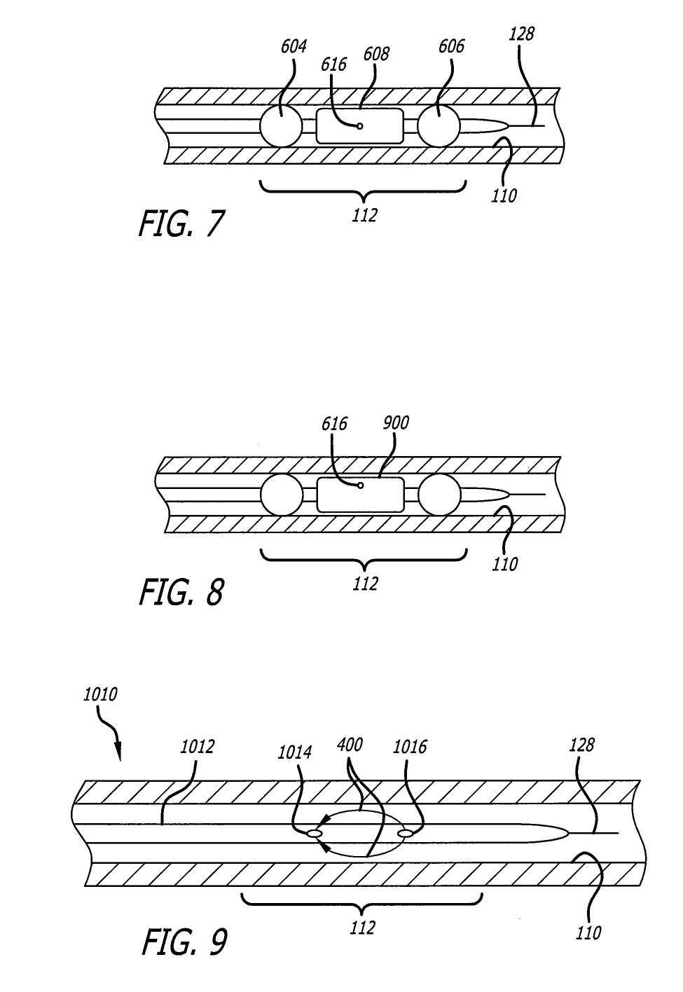

Referring now to FIG. 7, in accordance with the invention, a reduction in the intravascular pressure within the occluded vessel segment 112 may be achieved in several ways. First, the agent delivery member 608 profile can be reduced by removing fluid from the agent delivery member inflation lumen 614. The reduction in profile will have a corresponding reduction in volume of the occluded vessel segment 112, thereby creating a reduction in pressure within that segment. Alternatively, fluid can be removed through the agent delivery lumen 616 thereby resulting in a decrease in fluid volume within the occluded vessel segment 112 and a corresponding reduction in intravascular pressure therein. In this way, intravascular pressure can be reduced below the normal diastolic pressure of the patient.

Referring to FIG. 8, after reducing the intravascular blood pressure, therapeutic agent 900 can be introduced through the agent delivery lumen 616. This can be done in separation or in combination with an increase in the agent delivery member 608 caused by introduction of an inflation fluid through the agent delivery member inflation lumen 614. The introduction of therapeutic agent 900 and inflation of the agent delivery member 608 will result in an increase in the intravascular pressure and a rapid urging of the therapeutic agent 900 into the relaxed intracellular gaps caused by the reduction in intravascular pressure. During the introduction of therapeutic agents, it is anticipated that the intravascular pressure will remain below the normal diastolic pressure of the patient. The lowering of intravascular pressure and introduction of therapeutic agent 900 into the occluded vessel segment 112 can be repeated as desired to achieve the intended delivery of therapeutic agents into the vessel wall.

Many alternative device embodiments can be contemplated by one of ordinary skill in the art to achieve the methodical steps necessary in order to deliver a therapeutic agent to a vessel 110 and improve the uptake of the therapeutic agent within the vessel wall, in accordance with this invention. For example, an alternative embodiment of an agent delivery device 1010 is shown in FIG. 9. The device includes an elongated catheter body 1012 having a proximal agent delivery port 1014 and a distal agent delivery port 1016 formed therein. The agent delivery ports are independent and disposed near the end of agent delivery lumens (not shown).

The agent delivery device 1010 can be delivered over a guidewire 128 to a treatment site within a vessel 110. By introducing a fluid under pressure through the distal agent delivery port 1016 while removing fluid through the proximal agent delivery port 1014, a fluid flow can be established within the vessel 110. This fluid flow can result in a localized reduction in pressure below the normal diastolic pressure of the patient that will apply a suction to the surrounding vessel wall. The reduction in pressure applied to the vessel wall may cause the intracellular gaps to relax as described above. A therapeutic agent can then be delivered into the vessel 110 where it will be taken up by the vessel wall.

Further to this embodiment, the flow that is established may have an additional beneficial effect on vascular uptake of the therapeutic agent. The endothelial cell structure is generally understood to have an overlapping morphology in which the downstream cells are partially covered by the upstream cells. If a reversed flow is established within the blood vessel, a drag force on the cells may cause these overlaps to flex in an opposite direction from their normal orientation, which will further increase the intracellular gaps. As with the effect of lowered intravascular pressure therefore, reverse flow within the vessel 110 may lead to improved uptake of therapeutic agents within the intracellular gaps.

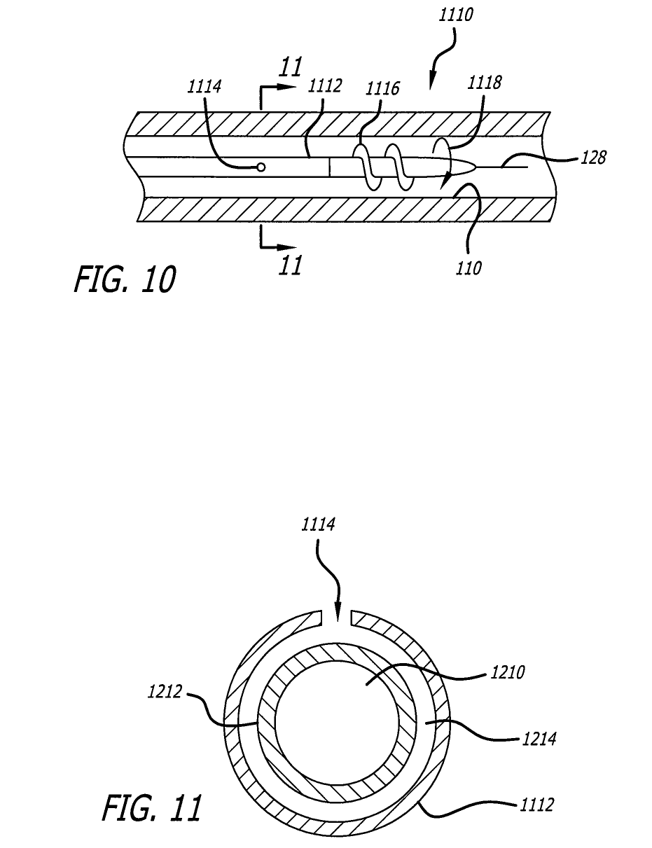

Referring to FIG. 10, yet another embodiment of a drug delivery device comprises a catheter body 1112 with an agent delivery port 1114 formed therein proximal to the distal end of the device. The device can be delivered over a guidewire 128 disposed within a guidewire lumen disposed within the device. A rotor 1116 is disposed distal to the catheter body 1112 and includes means for producing axial fluid flow through rotation 1118. The rotor 1116 is capable of rotating relative to the catheter body 1112 as rotational movement is developed by a drive cable 1212 that runs throughout the catheter body 1112. The drive cable 1212 can be rotated at high frequencies by attaching a drive motor unit (not shown). As the rotor 1116 rotates, axial flow is developed within the vessel 110. The flow causes a localized reduction in pressure that is translated into a suction on the vessel wall. Therefore, through varying the speed of the rotor 1116, the intravascular pressure near the rotor 1116 can also be controlled.

Referring now to FIG. 11 a cross sectional view of the drug delivery device of FIG. 10 is shown taken about line D-D, in accordance with this invention, therapeutic agents can be delivered from an agent delivery lumen 1214 disposed between the catheter body 1112 and the drive cable 1212 into the vessel 110 through the agent delivery port 1114. The guidewire lumen 1210 can be formed within the drive cable 1212.

In a further aspect of the alternative embodiment, a centering structure such as a cage can be formed around the rotor 1116 to prevent contact between the rotor 1116 and the vessel wall. This will further prevent damage to the patient anatomy. The cage structure may be expandable to allow it to be retrieved to a lower profile for delivery through the patient anatomy.

In still another aspect of the alternative embodiment, the rotor 1116 may comprise at least one spiral formed cable (not shown). The rotating cable may result in more random whipping within the vessel 110 that forms regions of turbulence and eddy currents, thereby producing a non-uniform change in the intravascular pressure. It is contemplated that non-uniform changes in the intravascular pressure could create localized changes in the size of the intercellular gaps in accordance with this invention.

The invention described herein provides devices and methods for delivering a therapeutic agent into a patient anatomy such as a coronary vessel and improving the uptake of the agent within the vessel wall by modifying the pressure that is applied to the vessel wall. The reduction in intravascular pressure that this invention provides in combination with the delivery methods takes advantage of the patient anatomy to achieve a more efficacious therapy.

It will be appreciated that in practice it can be difficult to totally isolate a vessel section. Vessel side branches, capillaries, and vaso vasorum communicate with the main vessel over its length. In this situation, the present invention is envisioned to function without complete vessel occlusion. Since the mechanism of reducing vascular pressure with these methods does not require complete isolation of the vessel to realize a change of vascular pressure, it will be appreciated that the invention can function in both completely and partially isolated vessel segments.

While several embodiments of the invention have been illustrated and described, it will be appreciated that various changes can be made therein without departing from the scope of the invention. For example, it is possible to modify the position of the agent delivery ports or the direction of established flow as described above and in the figures while still improving the delivery of therapeutic agents in accordance with this invention. Further, the pressures described above may be modified, for example, by increasing the upper pressure during the delivery cycle to a pressure above diastolic pressure, while still improving delivery of therapeutic agent in accordance with this invention. To this effect, it is contemplated that other pressures could be used. For example, the upper and lower pressures of the cyclic pressure delivery method may be other than the suggested values above. It is possible to achieve improved uptake of therapeutic agent using cyclic pressure ranges above normal diastolic pressure, as well.

Many other modifications can be made within the scope of this invention without departing from the intent or the essence of the invention. It is therefore intended that the scope of the invention be determined from the following claims and equivalents thereof.

What is claimed:

1. A medical device comprising:
a catheter body including a proximal end and a distal portion;
a first sealing member and a second sealing member disposed on the catheter body distal portion, the first sealing member adapted to seal against a first location and the second sealing member adapted to seal against a second location disposed distal to the first location, thereby forming a defined space between the sealing members;
a first agent delivery lumen comprising a distal end disposed on the catheter body between the first and second sealing members, the first agent delivery lumen being adapted to remove fluid from the defined space, thereby reducing pressure within the defined space; and
a second agent delivery lumen independent from the first agent delivery lumen, comprising a distal end disposed on the catheter body between the first and second sealing members, the second agent delivery lumen being adapted to introduce fluid into the defined space.

2. The medical device of claim 1 further comprising:
a first inflation lumen disposed between the proximal end and the distal portion of the catheter body, the first inflation lumen being in fluid communication with the first sealing member; and
a second inflation lumen disposed between the proximal end and the distal portion of the catheter body, the second inflation lumen being in fluid communication with the second sealing member.

3. The medical device of claim 2 wherein the first and the second sealing members comprise compliant balloons.

4. The medical device of claim 1 further comprising a guidewire lumen disposed within the catheter body, whereby the medical device can be delivered over a guidewire into a patient anatomy.

5. The medical device of claim 1 wherein the first agent delivery lumen is larger than the second agent delivery lumen.

6. The medical device of claim 1 wherein the distal end of the first agent delivery lumen is proximal to the distal end of the second agent delivery lumen.

7. The medical device of claim 1 wherein the first agent delivery lumen has a diameter of between 500 micrometers and 1 millimeter.

8. A medical device comprising:
a catheter body including a proximal end and a distal portion;
a first sealing member and a second sealing member disposed on the catheter body distal portion, the first sealing member adapted to seal against a first location and the second sealing member adapted to seal against a second location disposed distal to the first location, thereby forming a defined space between the sealing members;
a first agent delivery lumen comprising a distal end disposed on the catheter body between the first and second sealing members, the first agent delivery lumen being adapted to remove fluid from the defined space, thereby reducing pressure within the defined space;
a second agent delivery lumen independent from the first agent delivery lumen, comprising a distal end disposed on the catheter body between the first and second sealing members, the second agent delivery lumen being adapted to introduce fluid into the defined space;
a first inflation lumen disposed between the proximal end and the distal portion of the catheter body, the first inflation lumen being in fluid communication with the first sealing member; and
a second inflation lumen disposed between the proximal end and the distal portion of the catheter body, the second inflation lumen being in fluid communication with the second sealing member.

9. A method of delivering an agent into a patient anatomy comprising:
introducing a medical device into a vessel of the patient anatomy, the medical device comprising a catheter body including a proximal end and a distal portion, a first sealing member and a second sealing member disposed on the catheter body distal portion, a first agent delivery lumen, the first agent delivery lumen with a distal end disposed on the catheter body between the first and second sealing members, a second agent delivery lumen independent from the first agent delivery lumen, comprising a distal end disposed on the catheter body between the first and second sealing members, a first inflation lumen disposed between the proximal end and the distal portion of the catheter body, the first inflation lumen being in fluid communication with the first sealing member, and a second inflation lumen disposed between the proximal end and the distal portion of the catheter body, the second inflation lumen being in fluid communication with the second sealing member;
sealing the first sealing member against the first location by introducing an inflation fluid through the first inflation lumen;
flushing the vessel distal to the first sealing member by introducing a first agent through the first agent delivery lumen;
sealing the second sealing member against the second location by introducing the inflation fluid through the second inflation lumen, thereby forming a defined space;
introducing a second agent into the second agent delivery lumen;
removing fluid from the first agent delivery lumen, thereby decreasing the pressure within the defined space; and
introducing the second agent into the defined space through the second agent delivery lumen, whereby the second agent is delivered into the patient anatomy.

10. The method of claim 9 wherein the first and second agents are the same.

11. The method of claim 9 wherein the first agent comprises a flushing agent.

12. The method of claim 9 wherein the second agent comprises a therapeutic agent.

13. The method of claim 9, wherein removing fluid from the first agent delivery lumen decreases the pressure within the occluded vessel section to about 10 mm Hg.

14. The method of claim 9 wherein introducing a second agent into the second agent delivery lumen increases the pressure within the occluded vessel section to about 60 mm Hg.

15. A medical device comprising:
a catheter body comprising a proximal end, a distal portion, and at least three lumens disposed therebetween;
an agent delivery member disposed on the catheter body distal portion and being in fluid communication with a first inflation lumen, the first inflation lumen being one of the at least three lumens disposed between the proximal end and distal portion;
an agent delivery lumen capable of delivering a therapeutic agent through the catheter body and the agent delivery member, the agent delivery lumen being one of the at least three lumens disposed between the proximal end and distal portion;
a first sealing member disposed on the catheter body near the distal portion and proximal to the agent delivery member;
a second sealing member disposed on the catheter body near the distal portion and distal to the agent delivery member; and
a second inflation lumen in fluid communication with the first sealing member and the second sealing member, the second inflation lumen being one of the at least three lumens disposed between the proximal end and distal portion.

16. The medical device of claim 15 wherein the first and second sealing members comprise compliant balloons.

17. The medical device of claim 15, further comprising a guidewire lumen disposed within the catheter body, the guidewire lumen being adapted to facilitate tracking of the catheter body over a guidewire.

18. The method of claim 12, wherein the therapeutic agent is selected from the group consisting of anti-proliferatives, cytostatics, anti-inflammatories, antineoplastics, antiplatelets, anti-coagulants, anti-fibrins, antithrombotics, antimitotics, antibiotics, antiallergics, and antioxidant compounds.

19. The method of claim 18, wherein the cytostatic agent is selected from the group consisting of everolimus, ABT-578, tacrolimus, and pimecrolimus.
